(19) United States
(12) Patent Application Publication
(10) Pub. No.: US 2014/0243828 A1
(43) Pub. Date: Aug. 28, 2014

(54) Orthopaedic Device

(71) Applicant: Jake P. Heiney, Lambertville, MI (US)

(72) Inventor: Jake P. Heiney, Lambertville, MI (US)

(73) Assignee: Jake P. Heiney, Lambertville, MI (US)

(21) Appl. No.: 14/238,266

(22) PCT Filed: Aug. 7, 2012
(86) PCT No.: PCT/US12/49834

§ 371 Date: Apr. 1, 2014

Related U.S. Application Data
(60) Provisional application No. 61/522,452, filed on Aug. 11, 2011.
(60) Provisional application No. 61/545,783, filed on Oct. 11, 2011.

(51) Int. Cl. A61B 17/80; A61B 17/17

(52) U.S. Cl. CPC A61B 17/8085; A61B 17/8057; A61B 17/1728
USPC 606/70; 606/96; 606/281

(57) ABSTRACT

An orthopaedic surgical system and method for holding a surgical tool in a desired position during an orthopaedic surgical procedure.

### BACKGROUND OF THE INVENTION

This invention relates in general to orthopaedic surgery and in particular to a device for enhancement of orthopaedic surgery.

Orthopaedic treatment of fractures dates back to Egyptian times which included external fixation of fractures. In 1565, there was the first report of plate fixation for a cleft palate. In 1775, internal cerclage wiring was developed. In 1885, the Hannsmann plate developed in which the bent portion protruded from the soft tissues. In 1894, Sir William Lane described an all inside screw fixation of a tibia fracture, and added using plates in 1905. In 1912, Sir Robert Jones, stated that open reduction internal fixation offered the best functional outcomes for fracture fixation. In the 1920s, internal fixation popularity declined because of high rates of infection. In the 1940s, antibiotics and surgical asepsis technique were developed and dramatically decreased infection rates.

In 1949, Robert Danis “father of modern osteosynthesis” developed internal compression plate fixation with open reduction internal fixation as the only method of fracture treatment that allows complete restoration of anatomy. In 1958, the Swiss AO group developed the modern day principals of fixation which are anatomic reduction, stable internal fixation, preservation of blood supply, and early, active pain-free mobilization. In 1969, the Dynamic Compression Plate (DCP) was developed. In the 1980s, Brunner and Weber developed the wave plate in order to decrease vascular disruption. Also in the 1980s, Heitemeyer and Hierholzer introduced the idea of the bridge plate concept in which areas of comminution did not need absolute stability, but were instead “bridged” to preserve the vascular supply and allowed to heal en mass. The wave and bridge plate concepts were developed with the belief that minimal soft tissue disruption can be more important than anatomic reduction of bone fragments.

In 1992, Less-Contact, DCP Plate was developed in which bone porosis areas that were present because of disturbed circulation and contact pressure beneath plates were minimized by areas of less contact. In the 1990s, many simultaneous developments included Krettek et al, who introduced formal mini-invasive techniques with small incisions and submuscular tunnels. Mast introduced the Schuli nut with internal plating which allowed screws to lock into the plate and limited contact with bone, which made it a sort of “internal external fixator”. In the 1990s, Koval demonstrated the distal femur plate that was stronger than the blade plate. Perren developed a unicortical locked screw using the Morse taper. In Maxillo-facial and spine surgery, fixation without bicortical purchase was done which included using set screws and expanding heads. In extremity fractures a Less Invasive Stabilization System (LISS) was introduced in which fractures about the knee were fixed in a biologically sparing manner. Unique developments at that time with LISS included an insertion handle, anatomic pre-shaping from CT data, threaded screw heads (rather than Morse), larger screw core and percutaneous reduction tools. LISS had no ability for absolute stability (all relative stability) and no ability to angle screws

In 1994, the inflatable bone tamp was invented to reduce fractures with minimally invasively techniques. The inflatable bone tamp was developed commercially for the reduction of spine fractures while still having FDA indication for skeletal extremity fractures as well in the late 1990s. In the early 2000s, screw hole modifications were developed for internal fixation which included alternation of locking and non-locking holes in order to allow both relative and absolute stability in one implant. In 2003, the company Synthes introduced a merging of the tradition screw hole with a conical threaded hole to make a “combi-hole” Locking Compression Plate (LCP). In 2001, a single hole was developed to allow either non-locked screws, locking screws or pegs to be placed in the DVR (Distal Volar Radius) plating system by Orbay. Then poly-axial screws were developed to allow a degree of freedom when inserting screws, i.e. not forced to go in a single trajectory to lock, but at the same time allow the benefits of locked screws. During the 2000s, there was further development of external guides to allow percutaneous fixation with screws into the internal implants. Now polyaxial screw locking systems will accept both locking (several different mechanisms have been developed) and non-locking screws into the same hole and often have external guides to aid percutaneous placement of screws all over the skeletal system.

However, there is still a need for improved devices to further enhance orthopaedic surgery.

### SUMMARY OF THE INVENTION

This invention relates to an improved device to further enhance orthopaedic surgery

There is no skeletal fixation system that addresses the needs of the new potential percutaneous reduction tools, e.g. inflatable bone tamp, in order to use minimally disruptive techniques to the soft tissues. The present invention is a minimally disruptive fixation system to allow for fixation of fractures and a method for using the system. The system uses a multidirectional aiming guide to allow external to internal devices, such as, for example, a drill bit, a guide wire, a k-wire, an inflatable bone tamp and an injection needle, to be used and maintained in a way that does not exist presently. The minimally invasive plates and guides that are included in the present invention can be used with or without the multidirectional aiming guide as the surgeon sees fit. There are also easily moldable internal and external guides.

Therefore, it is the objective of this invention to allow for fixation of fractures in a minimally disruptive way to soft tissues and bone.

It is another objective of the invention that it allows several different external to internal devices to be guided and maintained in position as needed.

It is another objective that the invention be able to be used anywhere in the body human or otherwise and that all inventions within may be made of any material including but not limited to plastic, carbon fiber, titanium alloy, stainless steel, etc.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1A is a sectional view of a bushing and retainer tube assembly that is in accordance with the present invention.

FIG. 1B illustrates the attachment of the assembly shown in FIG. 1 to a plate.

FIG. 2A illustrates an alternative embodiment of the assembly shown in FIG. 1A.

FIG. 2B illustrates another alternative embodiment of the assembly shown in

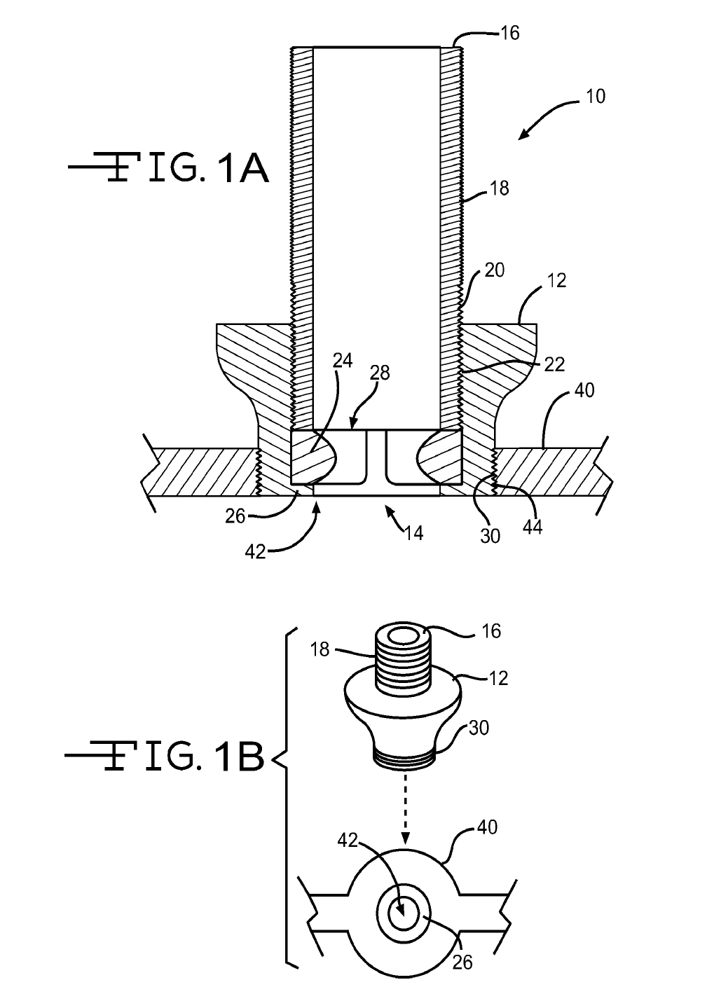

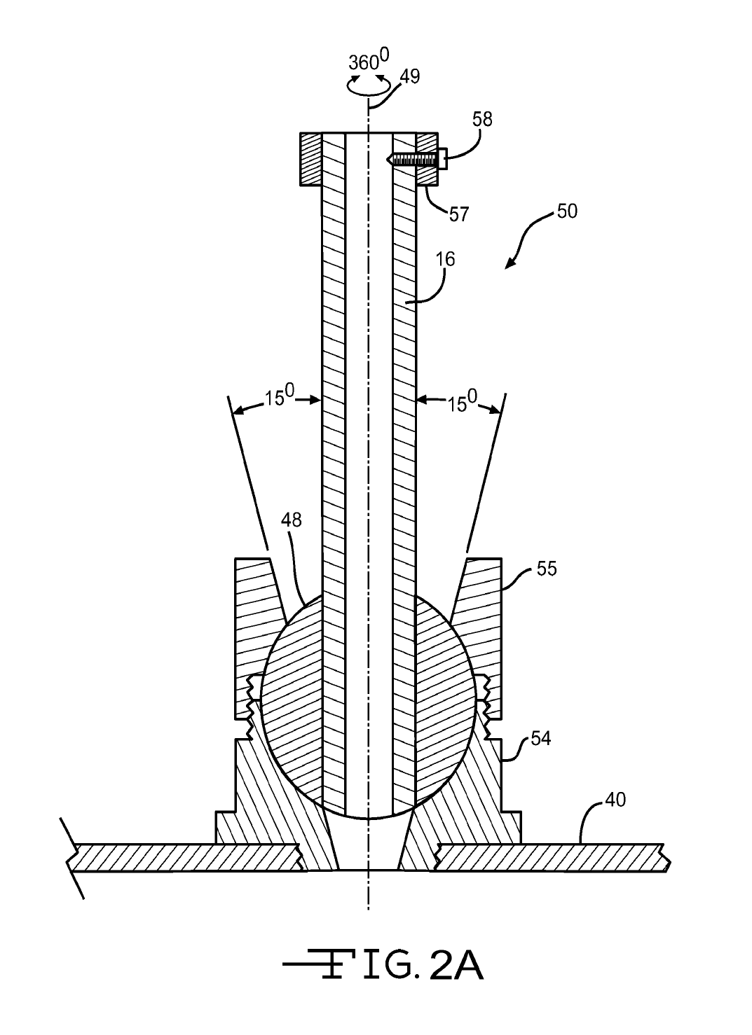

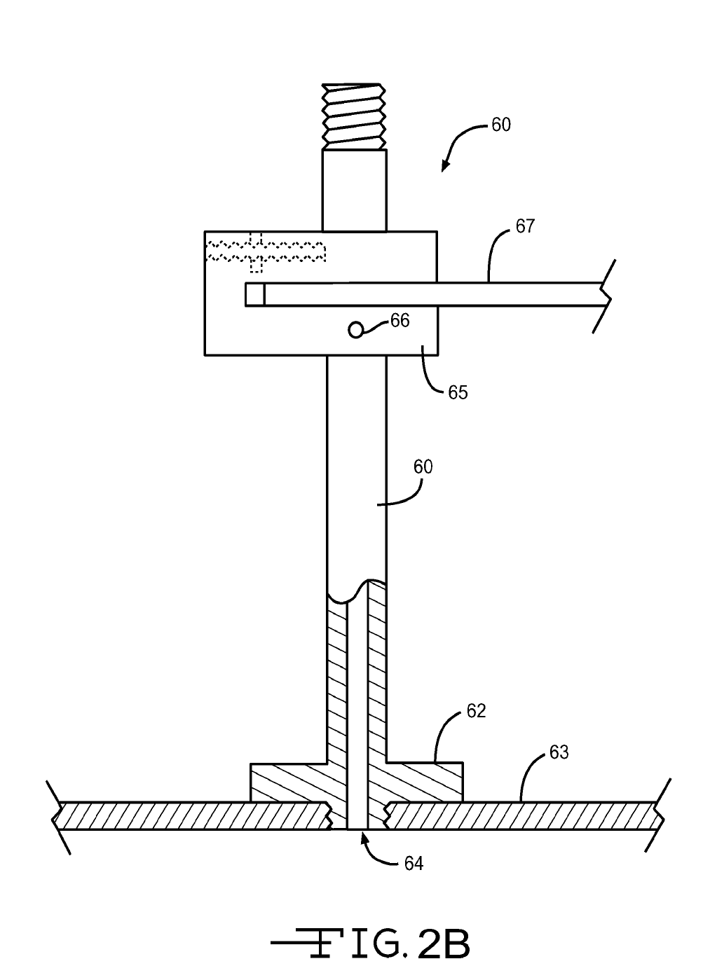

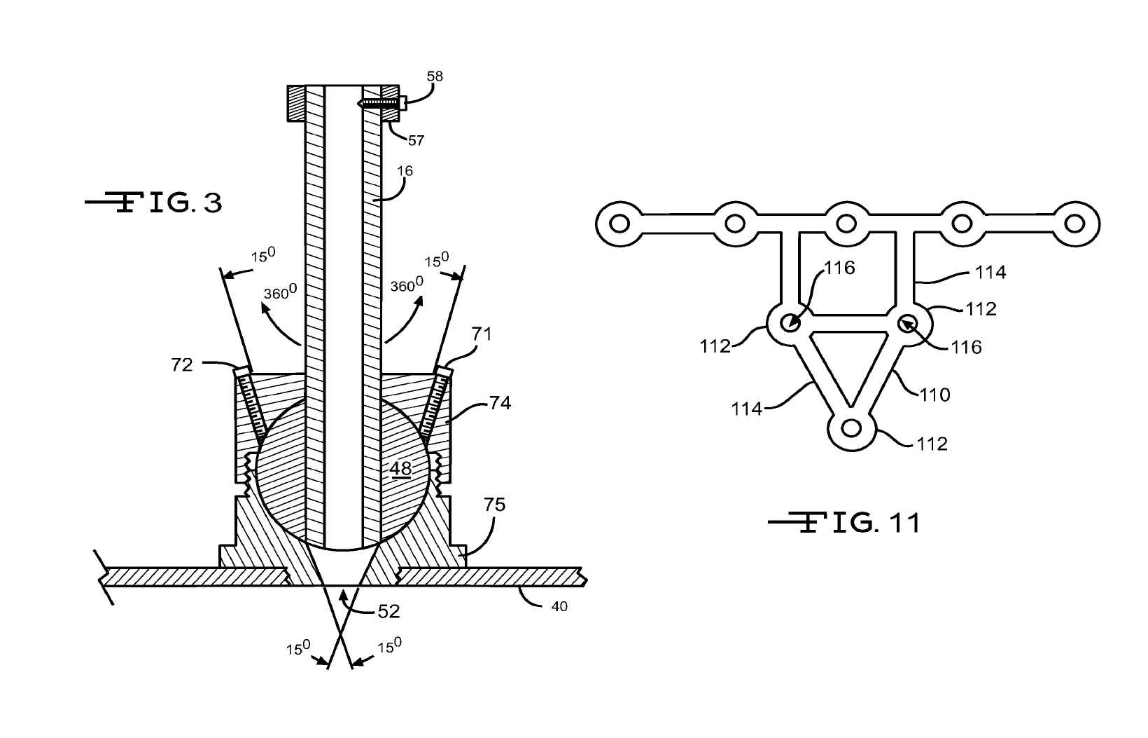

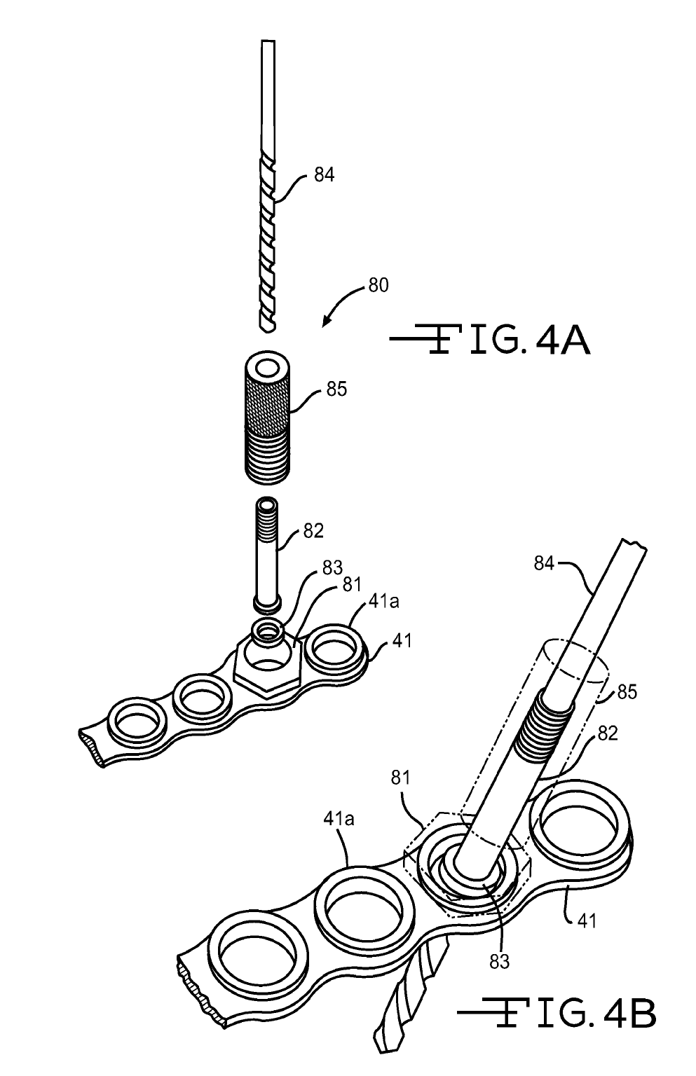

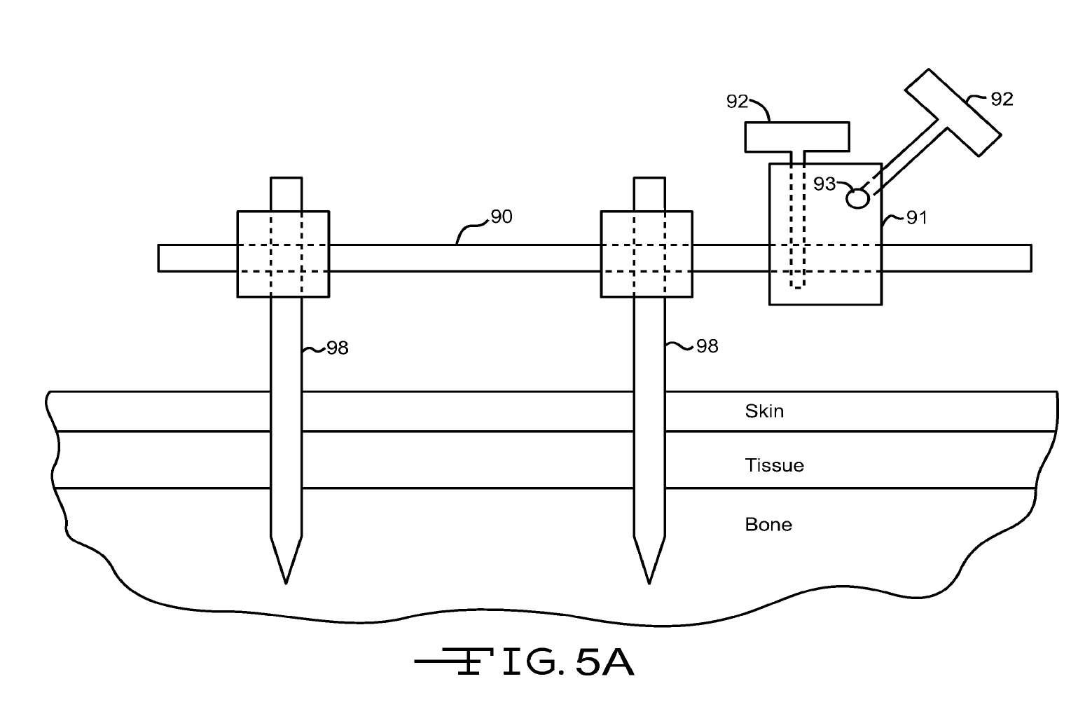

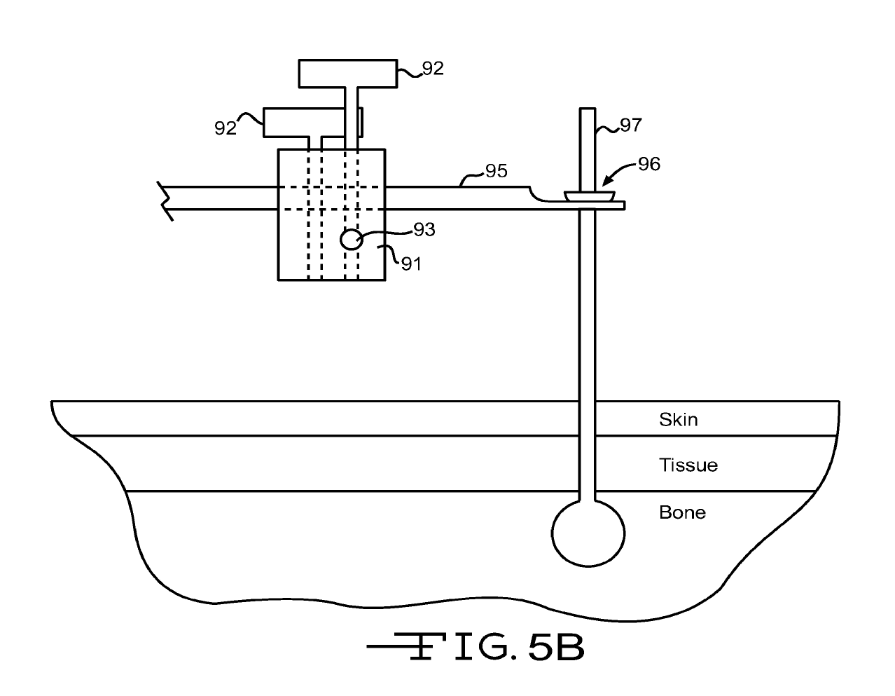

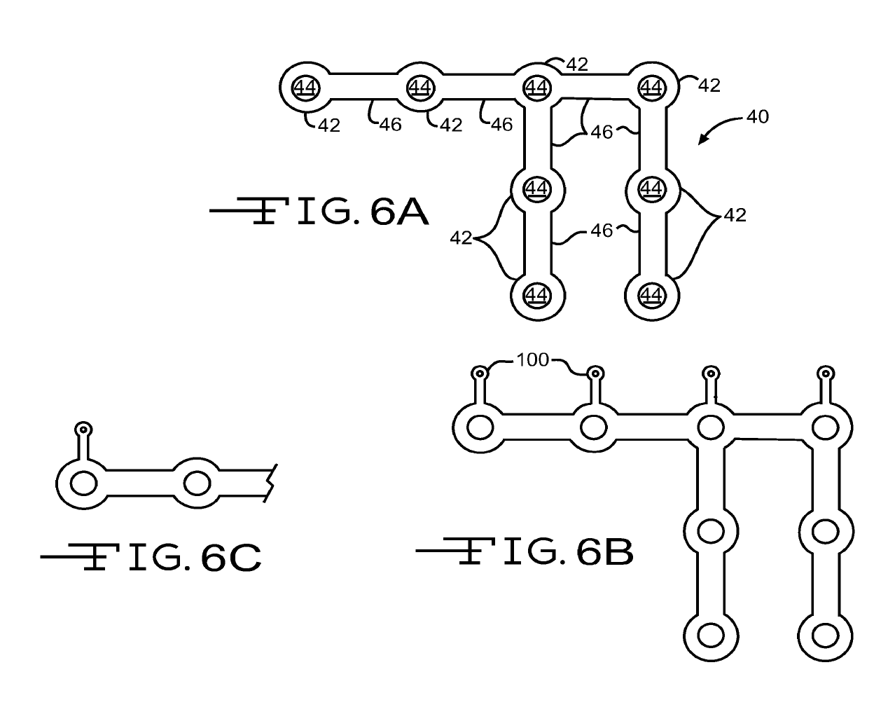

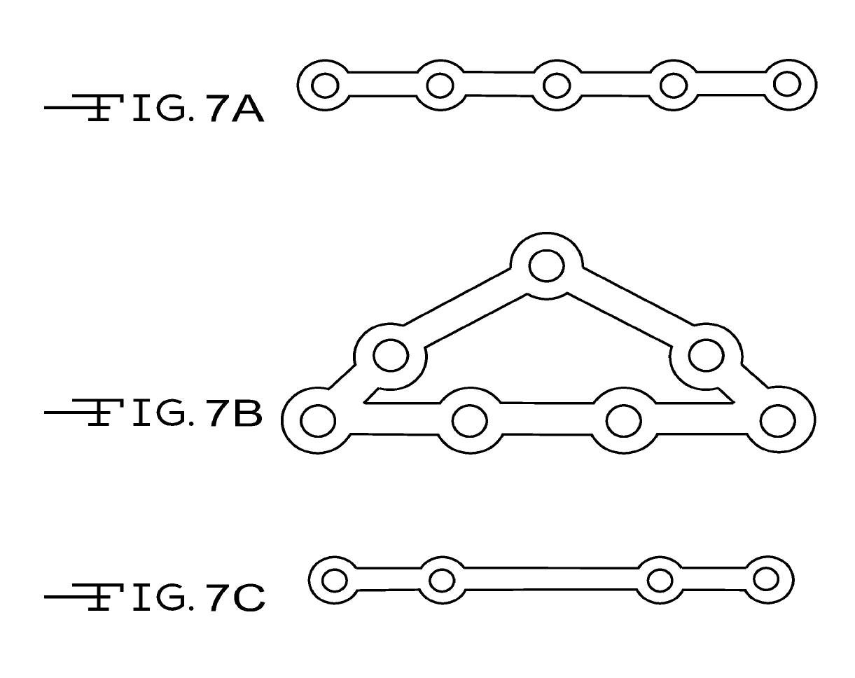

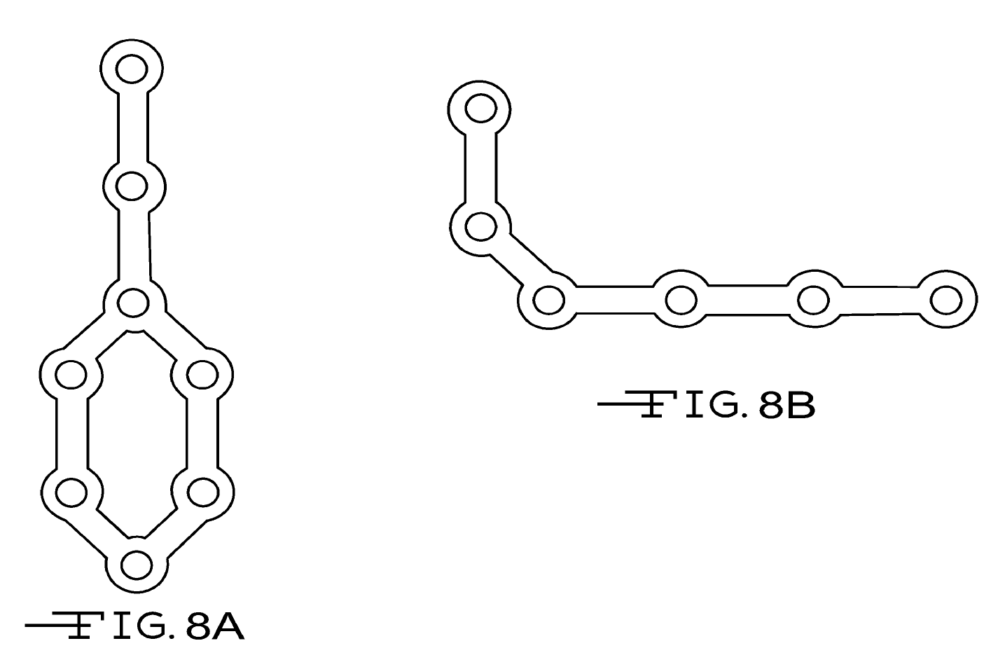

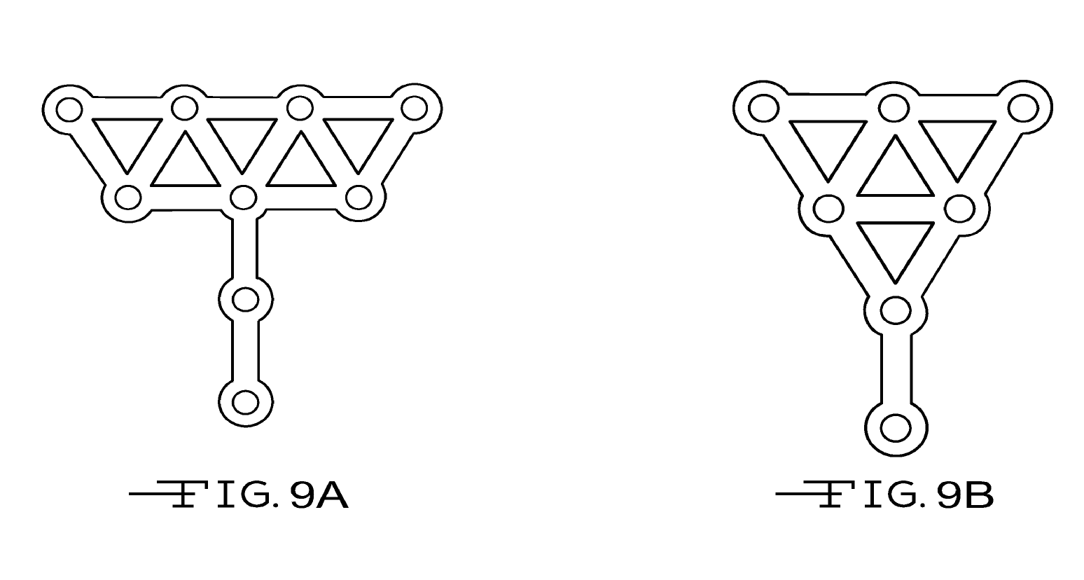

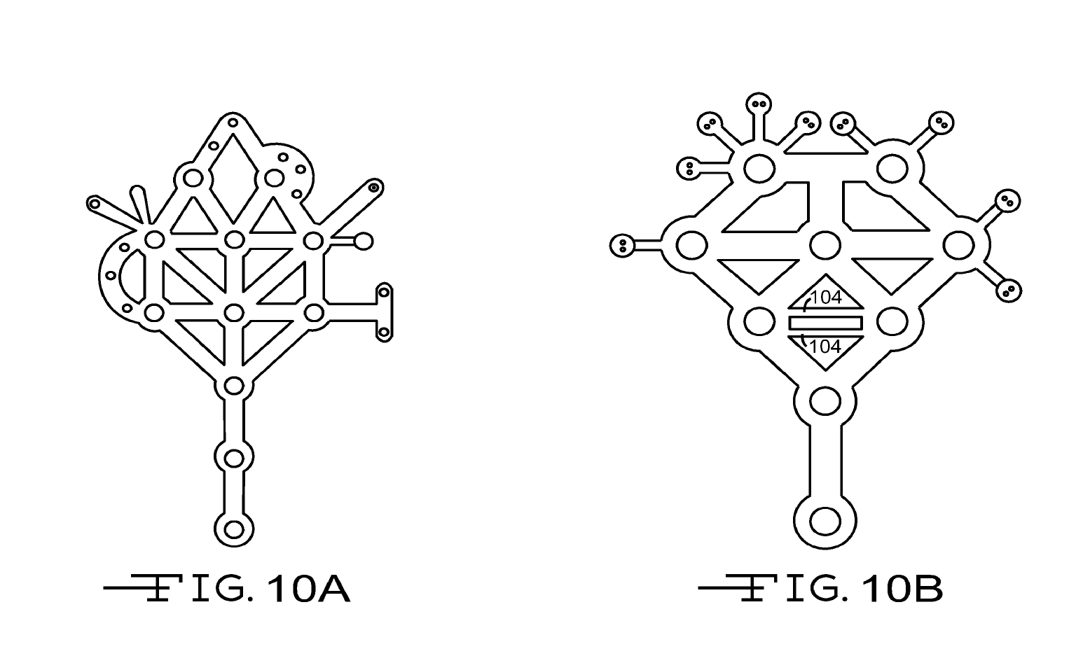

FIG. 1A for attachment of one plate to an additional plate to allow the more external plate to act as an external guide that is accordance with the present invention.

FIG. 3 illustrates another alternative embodiment of the assembly shown in FIG. 1A.

FIG. 4A—is an exploded view of yet another alternative embodiment of the assembly shown in FIG. 1A.

FIG. 4B is a partial phantom assembly view of the components shown in FIG. 4A.

FIG. 5A is a front view of an external fixation MAG adapter on external fixator (external fixator is prior art) that is in accordance with the present invention and that is used with the assembly shown in FIGS. 1-4.

FIG. 5B is an end view of the external fixation MAG adapter shown in FIG. 5A.

FIG. 6A shows an orthopaedic tibial plateau plate that is in accordance with the present invention and that is used with the assembly shown in FIGS. 1-4.

FIG. 6B shows an alternate embodiment of the orthopaedic tibial plateau plate illustrated in FIG. 6A that includes extensions for suture passing.

FIG. 6C illustrates the use of the suture passing extensions shown in FIG. 6B.

FIG. 7A shows an orthopaedic calcaneus plate that is in accordance with the present invention and that is used with the assembly shown in FIGS. 1-4.

FIG. 7B shows an alternate embodiment of the orthopaedic calcaneus plate illustrated in FIG. 7A.

FIG. 7C shows another alternate embodiment of the orthopaedic calcaneus plate illustrated in FIG. 7A. However, this plate has the intentional design of being used as an external guide on the posterior aspect of the calcaneus running in the longitudinal plane (sagittal plane).

FIG. 8A shows an orthopaedic medial tibial pilon plate that is in accordance with the present invention and that is used with the assembly shown in FIGS. 1-4.

FIG. 8B shows an orthopaedic anterolateral tibial pilon plate that is in accordance with the present invention and that is used with the assembly shown in FIGS. 1-4.

FIG. 9A shows an orthopaedic distal radius plate that is in accordance with the present invention and that is used with the assembly shown in FIGS. 1-4.

FIG. 9B shows another orthopaedic distal radius plate that is in accordance with the present invention and that is used with the assembly shown in FIGS. 1-4.

FIG. 10A shows an orthopaedic proximal humerus plate that is in accordance with the present invention and that is used with the assembly shown in FIGS. 1-4 and that include calcar reinforcement bars. FIG. 10A specifically demonstrates several different designs of potential suture hole layouts. Anyone of these could be chosen or a combination of them.

FIG. 10B shows another orthopaedic proximal humerus plate that is in accordance with the present invention and that is used with the assembly shown in FIGS. 1 through 4 and that include calcar reinforcement bars.

FIG. 11 illustrates an additional external guide that is in accordance with the present invention and that is used with the assembly shown in FIGS. 1 through 4.

FIG. 12A illustrates insertion of a plate cap that is in accordance with the present invention into a plate aperture .

FIG. 12B is a top view of the cap shown in FIG. 12A that has a star shaped recess formed therein.

FIG. 12C is a top view of the cap shown in FIG. 12A that has a hexagonal shaped recess formed therein.

FIG. 13A illustrates tools to lock the hex MAG in place.

FIG. 13B illustrates tools to turn the knurled tube and/or set screws included in the assembly shown in FIGS. 1-4 when not able to turn by hand and that is in accordance with the present invention.

FIGS. 14A through D illustrates several blunt submuscular clearing tools that are in accordance with the present invention and that are used with the assembly shown in FIGS. 1-4.

FIG. 15 illustrates plate cutters that for modification of plates found in FIGS. 6 through 10.

FIG. 16 illustrates cannulated screws and their guide wires that would be included, but not limited to these examples.

FIG. 17 illustrates solid screws and their star driver, but it is not limited to these examples (e.g. hexagonal screws and their driver).

FIG. 18 illustrates internal drill sleeves for the MAG. These sleeves are dropped into the proximal end of the tube (e.g. opposite the plate) according to the corresponding drill bit or k-wire or guide wire.

FIG. 19 illustrates a soft tissue protector for the use external to the system (prior art).

FIGS. 20A and B illustrate an external guide that has k-wire holes strategically placed from 2 views.

FIGS. 21A and B illustrate a “clip” that attached to cannulas that allow k-wires to pass through them.

FIGS. 22A and B illustrate a modified external guide with k-wire guide holes strategically placed and demonstrated in vivo.

### DEFINITION OF TERMS

External to Internal Devices are anything that pass from external of the tissues to internal. Examples include, but are not limited to, k-wires, inflatable bone tamps, drill bits, guide wires, etc.

Multidirectional Aiming Guide (i.e. MAG) is an external guide that has the ability to hold external to internal devices in a desired position once passed through the guide. They also have the ability to hold external aiming arms should there not be already made insertion points on the plate for that particular plate.

Multi-hole External Guide-Malleable, a cut-able guide that MAGs screw into (not implanted).

External Fixator “MAG” Adaptor that screws onto any external bars and then allows MAGs to be placed in position.

Internal Fixation Devices are anything implantable within a body (e.g. plates, screws, etc.)

External Interchangeable Guides are guides that can be used for external guides but at the same time can then be turned into implantable Internal Fixation Devices when desired.

Minimally Disruptive Fixation System (i.e. MDFS) denotes the complete present invention or whole system which together includes MAGs, external guides, MAG adaptor, and internal fixation that is compatible with the MAGs.

### DETAILED DESCRIPTION OF THE PREFERRED EMBODIMENT

The present invention is directed toward an orthopaedic device, a Multi-Directional Aiming Guide (MAG) that receives a surgical instrument and allows degrees of freedom. Once the surgical instrument has been placed in a desired position, the MAG can then be locked into the position. The device provides up to three degrees of freedom of movement. Thus, it is the intent of the inventor that the illustrations herein are merely several embodiments of a multitude of different mechanisms that are intended to accomplish the inventor's end of the total concept. The end unique invention concept being three different abilities of the MAG to lock, which include.

One, the ability to lock the MAG into a position with a first mechanism, such as on a plate or external guide or fixator adaptor.

Two, the ability to have an second, internal, mechanism within the MAG that allows for a range of variable degrees of angulations thru the MAG and then have the ability to lock that position (i.e. angle) so that it does not change while the user works.

Three, the ability to then pass external to internal devices through the MAG and allow a third mechanism of the MAG to lock that device and maintain its position within the MAG.

The descriptions and illustrations that follow are for a few specific types of mechanisms that will accomplish these three goals, but the inventor's intent is to patent this idea and concept for any piece of orthopaedic equipment that can be fashioned in a multitude of ways to accomplish these goals. Thus, the illustrations are intended to be exemplary of the invention.

Referring now to the drawings, there is illustrated, in FIG. 1, a sectional view of Multi-Directional Aiming Guide (MAG) 10 that is in accordance with the present invention. The MAG 10 includes a generally cylindrical bushing 12 with a bushing aperture 14 extending there through. The bushing rotatably receives a hollow cylindrical retainer tube 16 The upper portion of the retainer tube 16 has a knurled outer surface 18 while a first set of threads 20 formed upon the outer lower surface of retainer tube 16 engage a corresponding second set of threads 22 formed on the upper interior surface of the busing aperture 14. The threads 20 and 22 cooperate with one another to advance the retainer tube 16 into the bushing aperture 14 when the retainer tube is rotated in a clockwise direction with respect to the bushing 12. In a similar manner, threads 20 and 22 cooperate with one another to retract the retainer tube 16 from the bushing aperture 14 when the retainer tube is rotated in a counter-clockwise direction with respect to the bushing 12.

The treaded portion 20 of the retainer tube 16 cooperates with inner threads 22 of the bushing 12 in a way that allows a range of motion or a range of motion may be obtained by opening and closing a variable opening aperture 28. This device, by either aperture or internal bearing, rotates in order to give a range of trajectory that can then be locked into position (external locking mechanism not shown on FIG. 1A, but could include an internal set screw, C-ring, etc.) Therefore, in total, there are three locking mechanisms within the invention.

A retaining device, or locking mechanism, 24 is disposed in the lower portion of the bushing aperture 14 adjacent to the lower end of the retainer tube 16. While the retaining device 24 is shown in FIG. 1 as being retained by a shoulder 26 formed at the lower end of the bushing aperture 14, the invention contemplates that other methods may be utilized to secure the retaining device 24 within the bushing aperture 14 (not shown). A variable diameter aperture 28 extends axially through the retaining device 28. Rotation of the retainer tube 16 in a clockwise direction urges the lower surface of the tube against the upper surface of the retaining device 24 to cause the diameter of the retaining device aperture 28 to be reduced and to clamp any object inserted through the aperture in a desired secure position. In a similar manner, rotation of the retainer tube 16 in a counter-clockwise direction moves the lower surface of the tube away from the upper surface of the retaining device 24 to cause the retaining device aperture 28 be increased and to release any object inserted through the aperture. Typical tools, or surgical instruments, that may be inserted through the bushing 14 during orthopaedic surgery include, for example, an inflatable balloon tamp, a Kirchner wire, and an orthopaedic drill. Alternately, other retaining devices may be provided, such as, for example, a chuck assembly or a collet (not shown).

The bushing 14 also includes a set of exterior threads 30 formed in lower exterior surface thereof. As also shown in FIG. 1, the invention contemplates that the bushing 12 is used in combination with an orthopaedic plate 40, which will be described below. As shown in FIG. 1, the lower portion of the bushing is received in an aperture 42 formed through the plate 40. The bushing exterior threads 30 co-operate with corresponding threads 44 formed on the surface of the plate aperture 42 to secure the bushing 12 to the plate, as illustrated in FIG. 1B. It is also contemplated that the bushing 12 is formed to allow a cannulated driver, such as, for example, a socket wrench, to fit over the bushing 12 and with clock-wise rotation inserting the outer treaded portion 30 of the bushing 12 into the threaded aperture 44 in a plate 40 and with counter-clockwise rotation retracting the bushing from the plate.

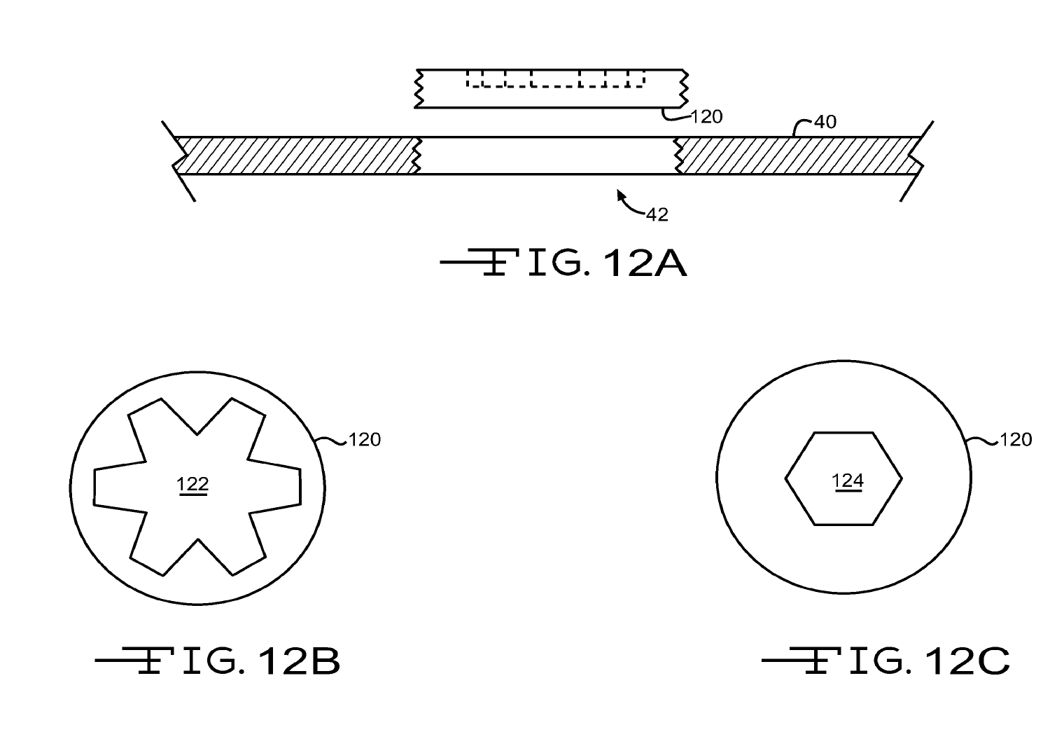

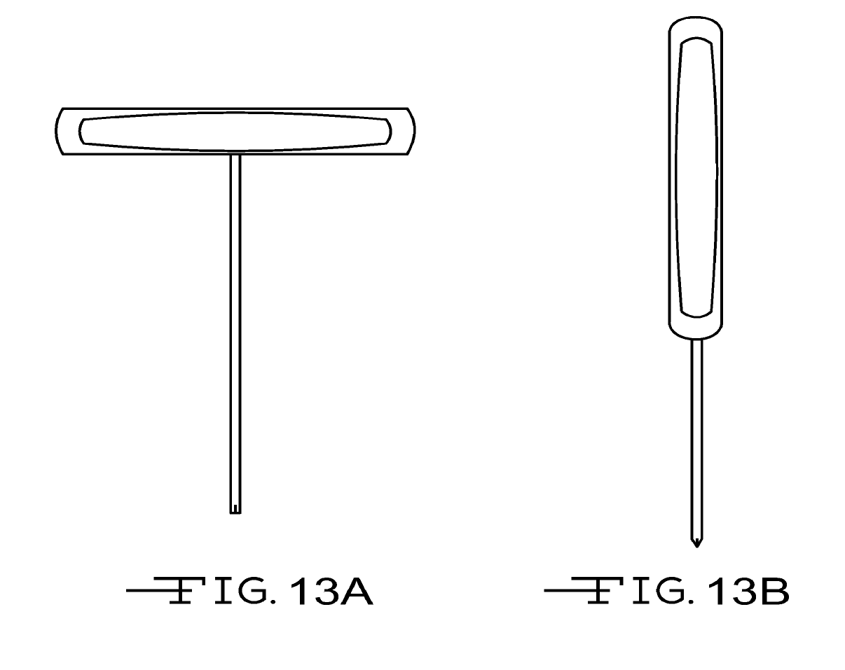

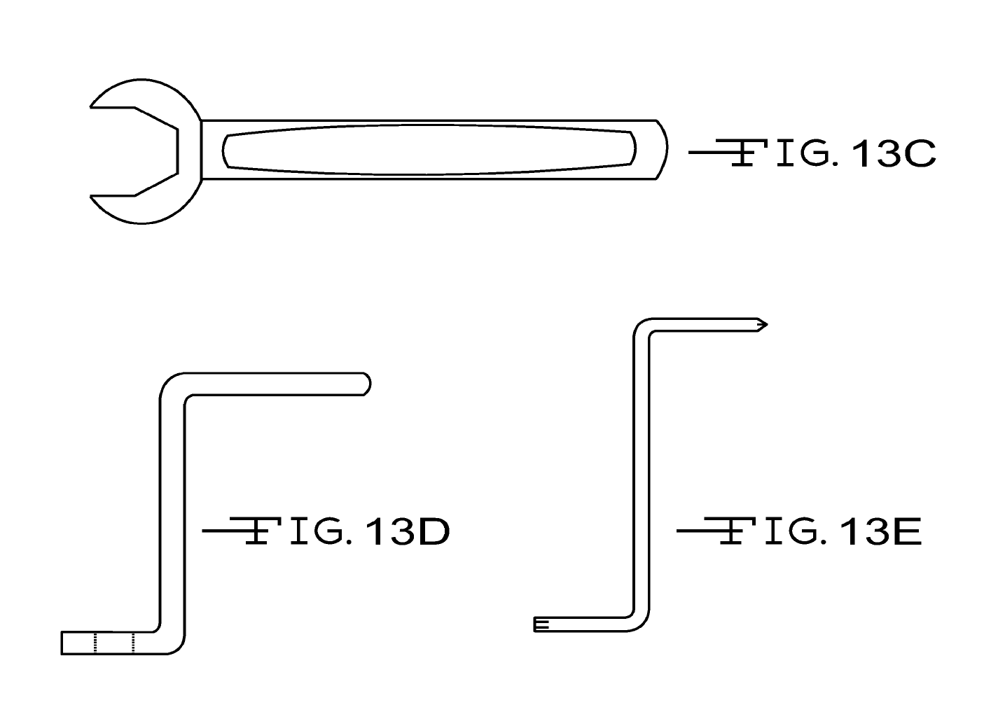

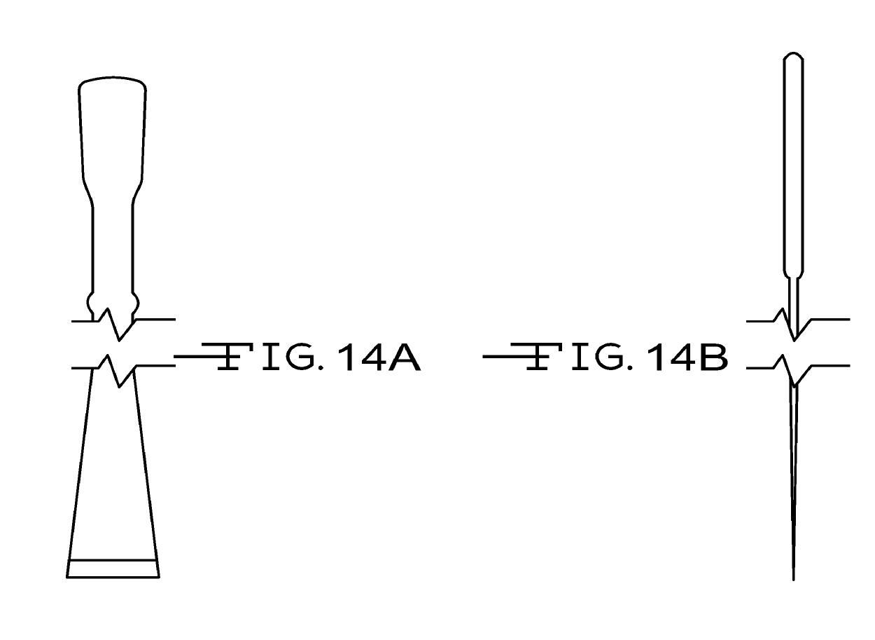

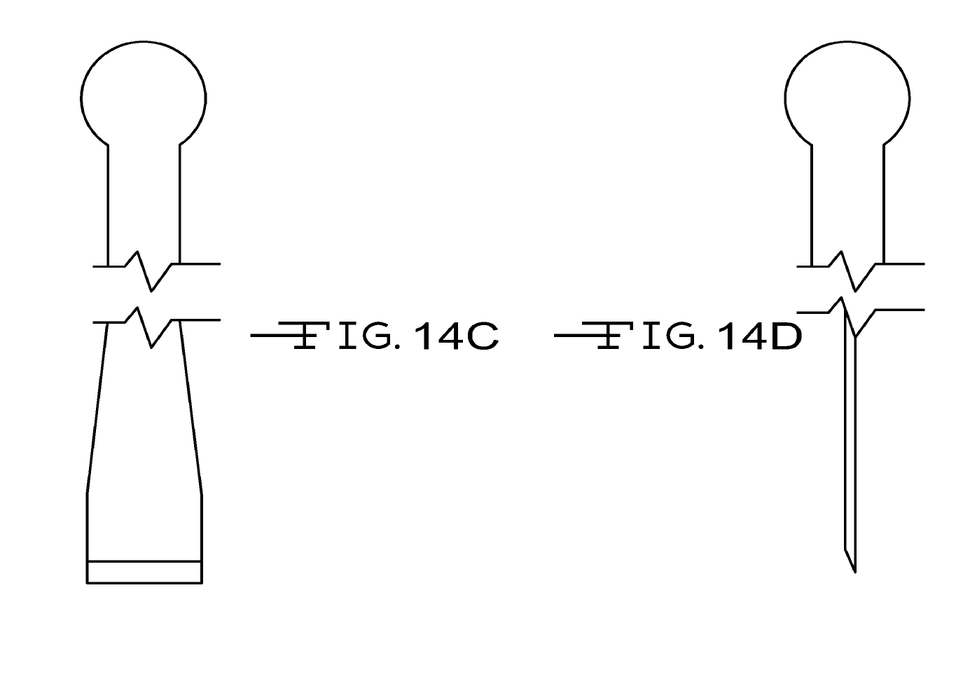

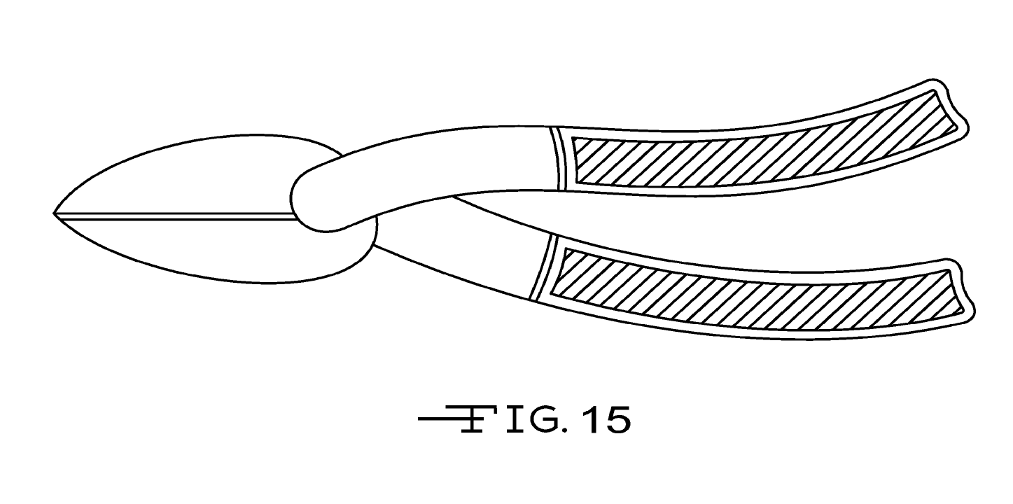

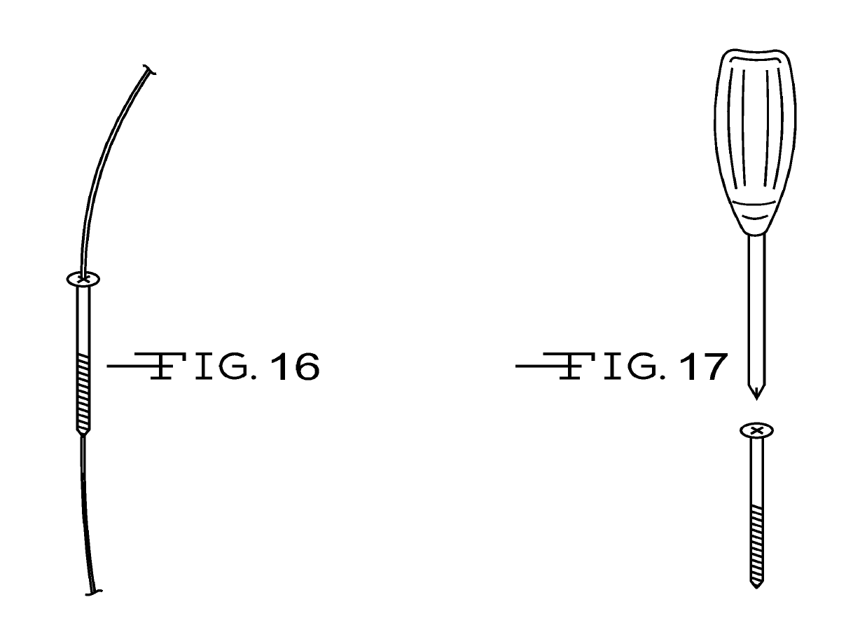

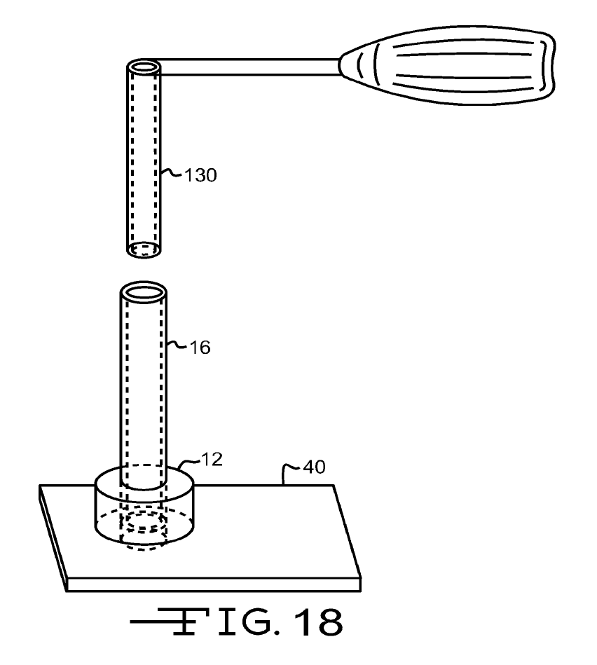

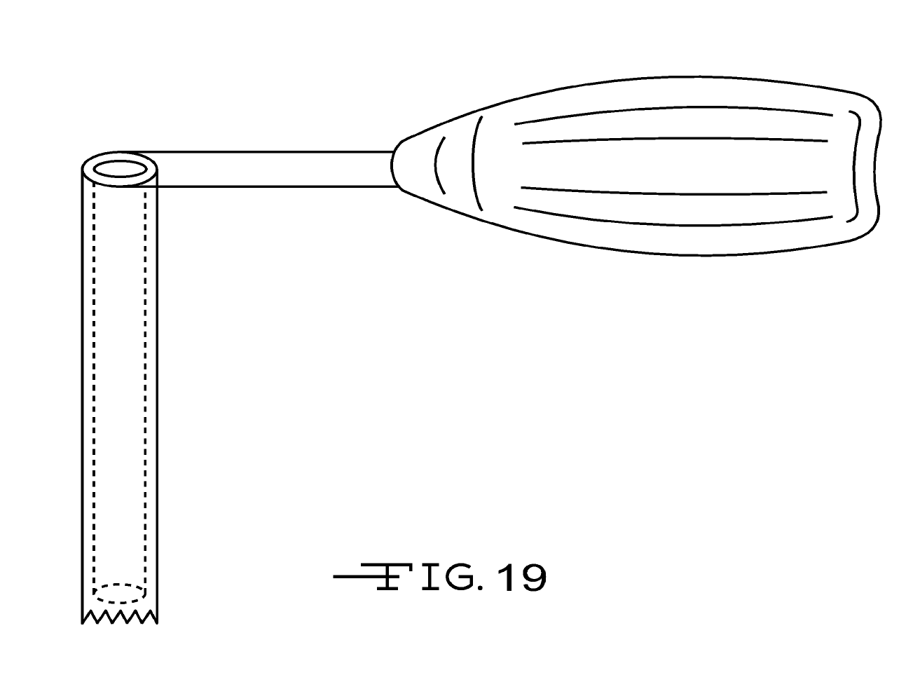

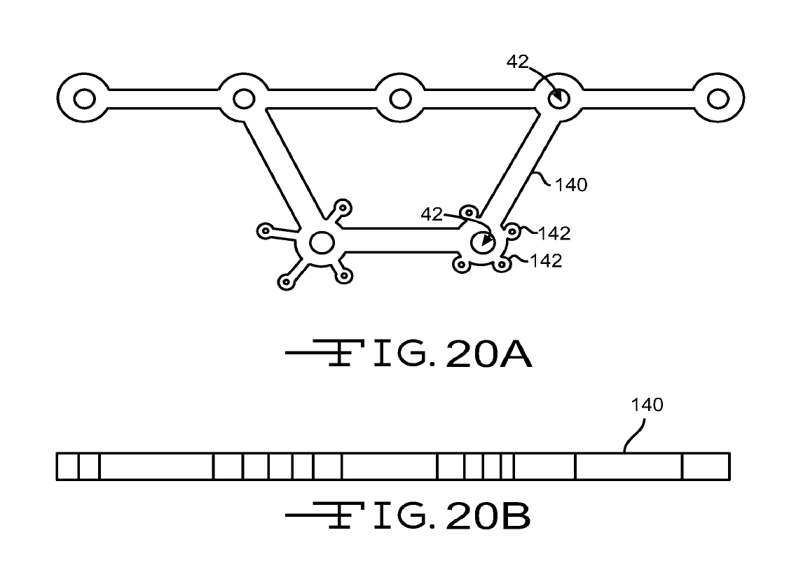

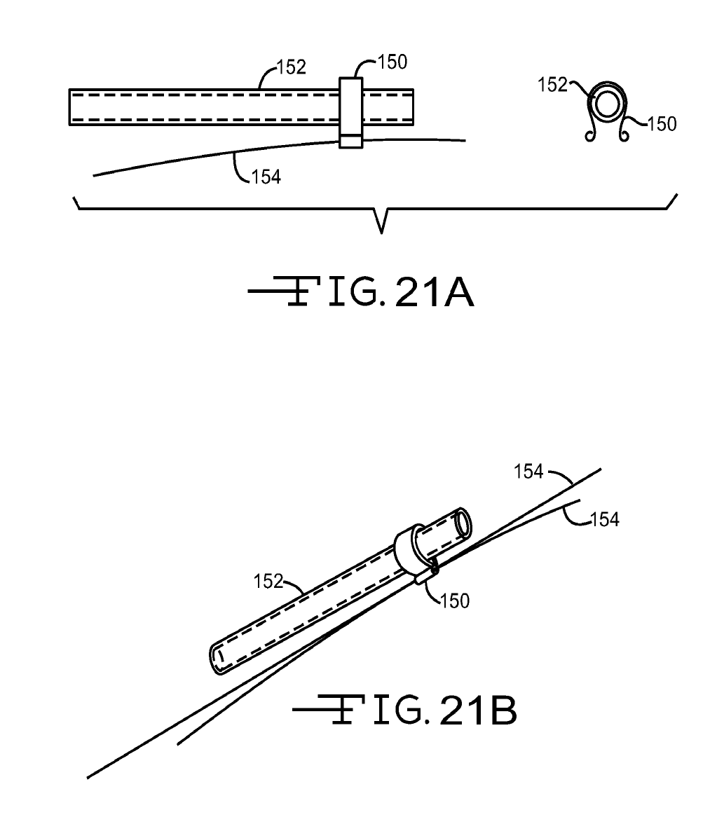

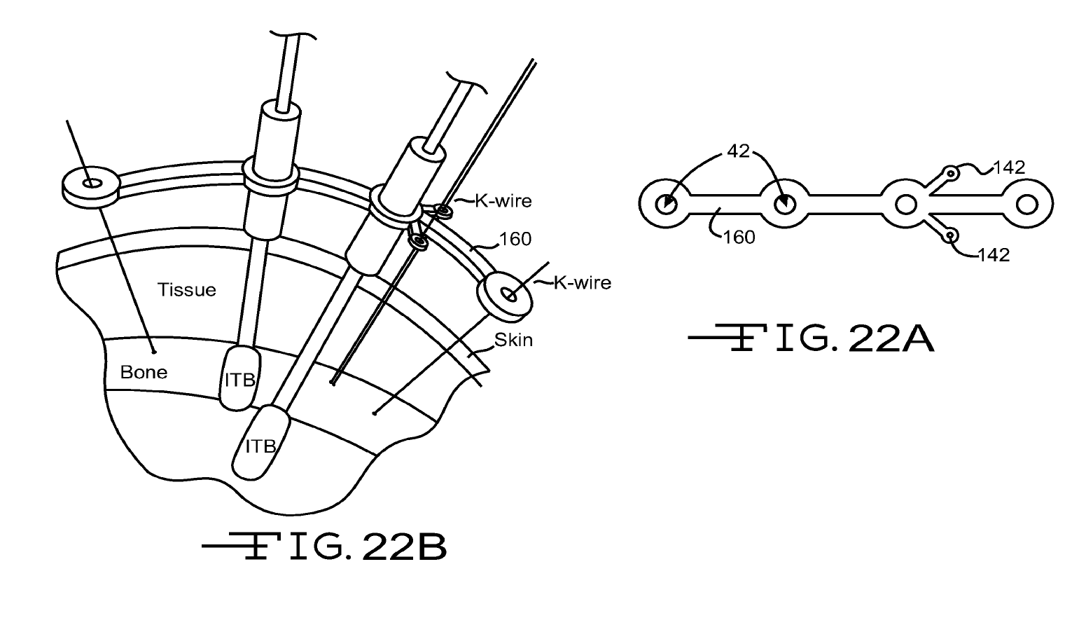

FIG. 2A illustrates an alternate embodiment 50 of the MAG shown in FIG. 1A. Components shown in FIG. 2A that are similar to components shown in FIG. 1 have the same numerical identifiers. As shown in FIG. 2A, the retainer tube 16 extends through a spherical positioner 48. The positioner 48 is received within a corresponding spherical void formed by screwing an upper bushing 55 into a lower bushing 54. Both upper and lower bushings 54 and 55 have a hexagonal shaped machined outer surface (not shown) that co-operates with a hex head wrench for rotating the bushings. As also shown in FIG. 2A, the lower bushing 54 is screwed into a threaded aperture formed through the orthopaedic plate 40. While threaded surfaces are shown for attaching the lower bushing 54 to the plate 40, it will be appreciated that the invention also contemplates utilizing other locking mechanisms (not shown). Initially, the upper bushing 55 is not fully tightened onto the lower bushing 54 in order to allow the retainer tube 16 and positioner 48 assembly to be are free to be rotated about a central axis 49 by 360 degrees while also being inclined to the central axis 49 by up to 15 degrees. While a maximum inclination of 15 degrees is illustrated in FIG. 2A, it will be appreciated that the invention also may be practiced with greater inclination angles by changing the aperture angles shown of the upper and lower bushings 54 and 55. Thus, the retainer tube 16 may be moved through a number of degrees of freedom. Once the desired position for the retainer tube 16, and any surgical instrument or other external device that may be extended through the retainer tube, is selected, the upper bushing 55 is further tightened into the lower bushing 54 to frictionally engage the positioner 48 and thereby lock the retainer tube 16 and positioner 48 assembly in the desired position and the portion of the lower bushing 54 that engages the plate 40 by a locking mechanism, which is shown as matching threads in FIG. 2A. The lower bushing 54 then engages the upper bushing 55 which can be tightened to lock the positioner 48 into position or loosened to unlock the positioner 48. When the positioner 48 is unlocked it can be further moved around in 360 degrees while allowing only the inclination range at the plate interface of approximate range, e.g. 30 degrees in all directions for the illustrated embodiment, for passing external to internal devices through the retainer tube 16.

The invention also contemplates the addition of a collar 57 that carries a set screw 58 that may be extended through a threaded aperture formed through the retainer tube 16 to engage an internal device (not shown) extending through the retainer tube. The internal device can then be locked in position within the retainer tube 58 by tightening the set screw 58 hence locking the external to internal device if desired, and thus controlling a third degree of freedom, i.e. the extent of the penetration of the external device through and beyond the retainer tube 16. It will be appreciated that the use of the collar 57 may be optional if the retainer tube wall is thick enough to allow secure insertion and retention of the set screw 58.

FIG. 2B illustrates another alternate embodiment 60 of the MAG in which there is. no ability to rotate the tube, but does include an allowance for wires to connect two plates together, an internal plate and an external that acts as an external guide in order to guide percutaneous work for holes that are under soft tissue. Wires would be the sizes of the tubes that could be inserted into bone via the modified MAG and then connected externally to the external plate. The MAG 60 shown in FIG. 2B includes a post 61 having is a hexagonal shaped machined base 62 that engages an internal plate 63 by a threaded connection. While a threaded connection between the shoulder 62 and the plate 63 is shown in FIG. 2B. It will be appreciated that other locking mechanisms (not shown) also may be utilized. As shown in FIG. 2A, the post 61 is a cannulated tube that allows external to internal devices to pass thru both the post and a plate aperture 64. A split collar 65 may be slid over the post 61 and can then be fixed at any distance from the shoulder 62 by engaging the locking mechanism 66. An external guide plate 67 that is an exact duplicate of the internal plate 63 that retains the post 61 provides an external guide for percutaneous work.

FIG. 3 illustrates another alternate embodiment 70 of the MAG shown in FIG. 2A. Components shown in FIG. 3 that are similar to components shown in FIG. 2A have the same numerical identifiers. Components 75 and 76 which are hexagonal machined parts that are similar to the upper and lower bushings 54 and 55 that are shown in FIG. 2A with component 75 engaging the plate 40 with a threaded connection or by a locking mechanism. The spherical positioner 48 is again the internal rotating mechanism that allows for the angulated range for locking through aperture 48. Once the correct position is found set screws 71 and 72 are tightened to lock in the position of tube 74. If the locking of the external to internal device is desired a setscrew 58 is again provided, with or without a collar 57, as a locking screw that is used as the third locking mechanism that has been described in the various embodiments.

FIGS. 4A and 4B illustrate an exploded view and a partial phantom assembled view of another alternate embodiment 80 of the MAG shown in FIG. 1A. Also shown is an external to internal device (i.e. a drill bit 84). FIG. 4A demonstrates a locking hexagonal head 81 bushing that tightens into a plate 41. It is noted that the plate 41 includes a circular lip 41a extending around the circumference of each of the apertures. An inner tube 82, which is a hollow internal sleeve, presses against a ring 83. The ring 83 has an accurate outer surface that allows the ring to rotate within the locking hex head 81. Also, the ring 82 rotates about and engages inside a knurled guide tube 83. The inner tube 82 is disposed within the guide tube 83 and extends into the ring 83. Once the desired angle is found, the guide tube 83 is threaded downward and engages both the ring 83 and the locking hex head bushing 81 to lock in the desired position. Once the knurled guide tube 83 is threaded downward, the threads are exposed on the inner tube 82 which would allow the attachment of a nut (not shown) as desired to lock the external to internal device, if locking within the inner tube 82 is desired.

FIGS. 5A and 5B illustrate front and end views, respectively, of an external fixator 90 with an attachment of an external fixator MAG adaptor 91 which provides a 360 degree adjustment range. The adaptor 91 is shown as having a cube shape that has apertures two holes 93 for external fixator rods 92 to pass through the adaptor. The external fixator rods 92 include T handles that can be tightened down when it is desired to lock the adaptor 92 onto the rod of the fixator 90. An extension 95 can then be placed through an orthogonal aperture 93 in the adaptor 91 to allow a MAG 96 to be attached to the extension. The MAG 96 is shown passing an external to internal device 97, which, in this case, is an Inflatable Balloon Tamp (IBT). Also illustrated in in FIGS. 5A and 5B, is the positioning of the assembly with pins 98 extending through skin and tissue and into bone. While a cube shaped adaptor 91 is shown in FIGS. 5A and 5B, it will be appreciated that the invention also may be practiced with adaptors having other shapes than a cube.

As indicated above, the present invention contemplates using the bushing 12 and retainer tube 16 assembly with an orthopaedic plate 40. The present invention also contemplates a number of enhanced orthopaedic plates that are illustrated in FIGS. 6 through 10, with a tibial plateau plate shown in FIG. 6, a calcaneus plate shown in FIG. 7, a tibial pilon plate shown in FIG. 8, a distal radius shown in FIG. 9, and a proximal humerus shown in FIG. 10. The invention also contemplates other plates for affixing to other bones that are not shown. The orthopaedic plates utilized with the system are designed to used with the MAG 10 and comprise a novel plating system that is extremely low profile and very easily bendable. Additionally, an open design allows IBTs to easily be used in conjunction with the plates. The invention contemplates that the thickness of the plates 40 are less than 1.0 mm at the webs and less than 1.4 mm at the threaded apertures. A plurality of threaded apertures 44 formed through the plates are intended to receive either MAGs or locking screws (not shown), as will be described below. Typical lengths of the plates include 56 mm, 65 mm, and 74 mm; however, plates with other lengths also may be used. Standard and locking screws are provided in diameters of 3.5 mm or 4.5 mm in lengths of 18 mm through 55 mm. It is also contemplated that standard and/or locking screws are provided in diameters of 3.5mm or 4.5mm in lengths of 18 mm through 55 mm are used with the plates 40.

Regarding the typical tibial plateau plate 40 illustrated in FIG. 6A, the plate consists of a plurality of discs 42 connected by narrow webs 46. As described above, the webs 46 are thinner than the discs 42 are easily bendable and cut, with the later characteristic allowing trimming of the plate to fit any particular situation. A threaded aperture 42 extends through each of the discs 40. It is contemplated that the plates are formed from surgical grade titanium; however, other materials also may be utilized to form the plates, such as, for example, stainless steel, plastic, titanium alloy and carbon fiber. It will be appreciated that the extensions 102 for passing sutures shown in FIGS. 6B and 6B may be applied to more of the discs than shown and to the other plates that are illustrated. The notation “MAGs” insert shown in the figures indicates the threaded apertures that are adapted to receive a MAG 10. Turning now to the plates illustrated in FIG. 10B, it is noted that calcar reinforcement bars 104 are disposed between several webs for retaining cartilage. Such bars may optionally be added to any of the plates that are included in the present invention.

The invention also contemplates a method of using the devices described above. First, a small stab incision is made through the tissue above the bone to be operated upon. Next, the tools shown in FIGS. 14A through 14D are used to clear off soft tissue as needed under the skin prior to plate placement. Then an appropriate plate is slid into position for the correct anatomic location, e.g. a tibial plateau plate as shown in FIG. 6, a calcaneus plate as shown in FIG. 7, a tibial pilon as shown in FIG. 8, a distal radius as shown in FIG. 9, or a proximal humerus as shown in FIG. 10,

The MAG allows the external to internal devices to be placed within a certain arc to be locked into position. Typical external to internal devices used with the MAG may include an inflatable bone tamp in order to obtain a reduction, a cannulated wire to a screw to be placed later over it, a drill bit to drill a path for the screw. The inventor believes that the knurled tube 16 included in the MAG, which may be for securing the external to internal device by tightening down, is unique to the system comprising the assembly shown in FIG. 1 and the plates shown in FIGS. 6 through 10. The present invention allows an IBT to be locked into place while it is inflated to solve a two fold problem present in the prior art, one, it is necessary to place the IBT around the plate and, two, that the IBT can move and not hold its position while being inflated.

Accordingly, one or more MAGs screw into the plate holes during procedures. As described above, once done with IBT, the MAGs could be drilled for a screw placement, or, if not using an IBT, then drill with a MAG for a locking screw or without a MAG for a non-locking screw, remove the MAG, if used, and place screw. The MAGs are then removed in order for the screw to be placed so that it can “lock” into the plate for those screws which will be locking. For non-locking screws, the screws can be drilled without the MAG, if desired, as they do not need a certain angle limit for locking, etc.

As shown in FIG. 1B, the MAG screws into any hole of the internal fixation plates 40 or external guides. The invention also contemplates utilizing two exact duplicate plates, with one located external from the patient and the other located internally within the patient. The external plate is then used as a guide for alignment with the internal plate with MAGS guiding k-wires placed through both of the plates. The inventor believes that this is a unique procedure. Once it is screwed into position, the MAG 10 allows external to internal fixation devices to pass within a certain defined multi-axial range. This range is limited in that anything beyond the circumference of the retainer tube 16 would not allow the locking mechanism 24 to work (similar to prior work of locking holes). Therefore additional range is not allowable (which would only be possible if the MAG is not screwed into position) with MAG in position. Once the external to internal device has been passed and it is in the position that is desired then the knurled tube is tightened this allows the locking mechanism, or internal bushing, 24 to tighten on the external to internal device and hence hold it in a desired position. Once the work is complete the MAG maybe unscrewed and, if desired, the knurled retainer tube 16 could be loosened to leave the internal to external device in position as the MAG is removed. Alternately, the knurled retainer tube 16 could be loosened and the internal to external device could be removed prior to the MAG being removed depending upon desired effect. The MAG can also allow all external devices, such as external guides to be attached via the MAG using the same mechanical mechanism as described.

Regarding FIG. 11, there is shown an external guide 110. Sometime it is necessary to place some screws parallel to each other and the external guide 110 is intended to aid in the placement. Similar to the plates 40 described above, the external guide includes a plurality of discs 112 connected to one another in a lattice of severable and/or breakable webs 114. Each of the discs 112 has a threaded aperture 116 formed there through that is adapted to receive and retain a MAG 10. In use, one or more MAGs are screwed into the discs threaded apertures 86 and k-wires are passed through the MAGS and percutaneously through the tissue to hold the guide 80. in place. Then additional MAGs are screwed into the other holes where it is desired to place screws into the underlying bone. Stab incisions are used to do the later operation.

FIG. 12A illustrates threaded caps 120 that may be screwed into the plate 40 threaded apertures 42 or, alternately, use other locking mechanisms for attachment to the plate. Because the plates 40 have such a low profile, the strength of the plates may be questioned in certain applications. If this should happen, the surgeons can insert the caps into the screw holes and make the plates stronger as desired. In theory, a surgeon could fill every hole with either a screw or a cap, according to the surgeon's choice. The caps 120 may have either a hex shaped recess 122 or a dtar shaped recess 124 formed in the upper surface, as shown in FIGS. 12B and 12C, respectively. The recess cooperates with the end of a corresponding tool to screw the cap 120 into a plate aperture 42. A the tool that includes either a traditional star or hexagonal head screwdriver that extends into holes formed in the top surface of the cap 120 is used to rotate the cap as it is screwed into the aperture 42.

The drawings that constitute a part of this specification include exemplary embodiments of the present invention and it will be appreciated that the invention also may be practiced other than shown in the drawings. While several specific examples of plating systems are given, e.g. tibial plateau, tibial pilon, calcaneus, distal radius, proximal humerus, the present invention is intended to work on any bone in the body. Therefore, the invention is for all skeletal bones. Henceforth, the concept of multidirectional aiming guides and the rest of the invention can be used to modify any existing or future internal or external fixation system.

FIGS. 13A through 13E illustrates tools utilized to turn the knurled retainer tube 16 included in the MAG 10 when a surgeon is not able to turn the tube by hand. FIGS. 14A through 14D illustrates blunt submuscular clearing tools that are used to clear soft tissue form bone before sliding plates 40 into position.

FIG. 17 illustrates solid screws and their star driver, but it is not limited to these examples (e.g. hexagonal screws and their driver).

FIG. 18 illustrates an internal drill sleeve 130 for the MAG. These sleeves are dropped into the proximal end of the tube (e.g. opposite the plate) according to the corresponding drill bit or k-wire or guide wire.

FIG. 19 illustrates a soft tissue protector for the use external to the system for completeness.

FIGS. 20A and B are plan and elevation views of an external guide 140 that is in accordance with the present invention. The external guide 140 includes a plurality of k-wire holes 142 strategically placed in a manner that will either deflect the IBT to go in the direction that the operator desires or allow the placement of guidewires for cannulated screws.

FIGS. 21A and B illustrate a clip 150 that attaches to any cannula 152 to allow k-wires 154 to pass in a strategic direction in order for the IBT to deflect and go in the proper direction. Also, the clip 150 can be used to place k-wires for which cannulated screws will pass over in the proper position. Other uses of the clip 150 can be used as imagined for k-wire placement.

FIG. 22 illustrates an external guide 160 placed on the outside of a bone that has k-wire guides to travel in the proper direction. This k-wire placement allows the blocking and deflection of IBTs into the proper position. It can also be used to place wires over which cannulated screws will pass in the proper direction. Other uses for k-wire guidance can also be imagined.

In summary, the Minimally Disruptive Fixation System (MDFS) is an orthopaedic fixation system unique in concept of being a minimally disruptive system for the reduction and fixation of fractures that uses several prior art concepts and builds on it with several unique properties and procedures. The MDFS includes a Multidirectional Aiming Guide (MAG) and orthopaedic or fixator plate. The MAG is unique in that it attaches to internal fixation (e.g. plates) or other external devices (e.g. external targeting template, or external fixator clamp adaptor) and allows for rotation (which can be locked in that angular position if desired) and then holds numerous different external-to-internal devices (e.g. drill bit, guide wire, k-wire, inflatable bone tamp, injection needle). The mechanism of the aiming guide is unique in allowing these devices to pass freely through an unlocked angular circumference and then hold the angle (lock) in position once the desired position is achieved for accurate placement as well as the ability to then hold the angle (locked) in position once the desired position is achieved for accurate placement and replacement of the external-to-internal devices.

The MDFS is unique in the concept of having holes and cut outs for the internal fixation portion (i.e. plates) made specifically for the Minimally Disruptive Fixation System. Reduction tools void filler delivery systems and implants can pass through or around these holes and cutouts easily for fracture reduction/fixation. Also, simple caps that can then thread into any holes in the plates to “fill” those holes as desired (e.g. to give plate more inherent strength).

Additionally, the MDFS that has a unique external multidirectional adapter that attaches to a distracter/fixator. It has the ability of free rotation and locking in place once the desired angle is achieved. This attaches to and uses the same Multidirectional Aiming Guide for external-to-internal device placement as exampled in FIGS. 1 through 4.

The MDFS with a mechanism in the Multidirectional Aiming Guide that is unique by the operation as follows. The base of the Multidirectional Aiming Guide threads into the desired internal fixation device hole or into the External Multidirectional Adapter with an external wrench, it then has the ability to freely move in a circumferential motion with an angle that can be locked into place once a desired position is achieved. This intraoperative locking ability is achieved by rotating the knurled cannulated handle until tight. This then locks the guide angle in place to allow the external-to-internal devices to maintain its position. The external-to-internal devices (e.g. IBT) can then be locked into position by either a mechanism within the MAG or just external to it.

The MDFS is also unique by having a Multi-hole External Guide which will allow the Multidirectional Aiming Guides (MAGs) to be attached. This device allows for the proper aiming and position maintenance for external-to-internal device placement and replacement. At least two MAGs can be used with locking K-wires to hold the Multi-hole External Guide in place while using the remaining MAG holes to work through. This external guide is moldable and cut-able to adapt to the external anatomy as needed.

Another feature of the MDFS that is unique in having the ability to allow external guides for implants to be attached when desired via MAGs or to allow the modified MAGs or MAGs to be used to attach two of the exact same plates together to guide percutaneous work.

In accordance with the provisions of the patent statutes, the principle and mode of operation of this invention have been explained and illustrated in its preferred embodiment. However, it must be understood that this invention may be practiced otherwise than as specifically explained and illustrated without departing from its spirit or scope.

What is claimed is:

1. An orthopaedic device comprising:
a bushing;
a retaining hollow cylindrical tube received by said bushing; and
a retaining device disposed within said bushing, said retaining device having an axial aperture formed therethrough that is adapted to receive a surgical tool, said retaining tube and retaining device being adapted to receive a surgical tool and allows at least one degree of freedom of movement for positioning said surgical tool, said retaining device co-operating with said retaining tube to lock said surgical tool in a desired position when said retaining tube is rotated in a first direction.

2. The orthopaedic device according to claim 2 wherein said retaining device also co-operates with said retaining tube to release said surgical tool in a desired position when said retaining tube is rotated in a second direction that is opposite from said first direction.

3. The orthopaedic device according to claim 2 further including a device for locking said surgical tool in a desired position within said retaining tube.

4. The orthopaedic device according to claim 3 wherein the device is a multidirectional aiming guide.

5. The orthopaedic device according to claim 4 wherein said multidirectional aiming guide is included in a minimally disruptive fixation system that includes at least one multidirectional aiming guide and at least one orthopaedic plate, said orthopaedic plate including a plurality of discs, each disc having an aperture formed there through that is adapted to accepts said multidirectional aiming guide and to fixate to it, said discs connected to one another by a web lattice that spaces said discs apart from one another to allow for working tools to be one of placed through said multidirectional aiming guide and placed in between the web lattice.

6. An orthopaedic device comprising:
a bushing;
a retaining hollow cylindrical tube received by said bushing; and
a rotational ring disposed within said bushing, said rotational ring being capable to rotate about an axis extending across said ring, said rotational ring and retaining tube being adapted to receive a surgical tool and allow at least one degree of freedom of movement for positioning said surgical tool, said rotational ring adapted to be locked in a selected position.

7. The orthopaedic device according to claim 6 further including a device for locking said surgical tool in a desired position within said retaining tube on the rotating ring

8. The orthopaedic device according to claim 2 said retaining device includes at least one of a compression nut, set screw and tapered lock disposed within said bushing and operative to lock said surgical tool in a desired position.

9. An orthopaedic plate comprising:
a plurality of discs, each disc having an aperture formed therethrough that is adapted to accepts a multidirectional aiming guide and to fixate to it; and
a web lattice connecting said discs to one another while spacing said discs apart from one another to allow for working tools to be one of placed through said MAGs and placed in between the lattice

10. The orthopaedic plate according to claim 9 further including at least one additional disc formed adjacent to one of the lattice discs, said additional disc having a aperture formed there through for receiving and passing a suture.

11. The orthopaedic plate according to claim 9 wherein the plate is formed from one of surgical grade titanium, stainless steel, plastic, titanium alloy and carbon fiber.

12. The orthopaedic plate according to claim 11 wherein the thickness of said plates is approximately 1.4 mm and the thickness of the webs is approximately 1.4 mm and further wherein said disc apertures are threaded, said threads adapted to co-operate with said multidirectional aiming guide to secure said multidirectional aiming guide to said plate.

13. A method for treatment of a bone fracture comprising the steps of:
(a) making a small stab incision through tissue in the vicinity of the bone fracture;
(b) clearing soft tissue form the fractured bone as needed;
(c) inserting a fixation plate through the incision;
(d) attaching at least one multidirectional aiming guide to the fixation plate;
(e) inserting a tool through the multidirectional aiming guide;
(f) tightening the multidirectional aiming guide to secure the tool in a desired position; and
(g) utilizing the secured tool to carry out the treatment procedure The method of claim 13 further including the step of repeating the previous steps as needed and completing fixation using tools and implants from a minimally disruptive fixation system as needed.

15. The method of claim 14 wherein the fixation plate is an orthopadaedic plate.

16. A method for treatment of a bone fracture comprising the steps of:
(a) making a small stab incision through tissue in the vicinity of the bone fracture;
(b) clearing soft tissue from the fractured bone as needed;
(c) placing an external guide in position adjacent to the incision;
(d) attaching at least one multidirectional aiming guide to the external guide;
(e) inserting a tool through the multidirectional aiming guide and into the incision;
(f) tightening the multidirectional aiming guide to secure the tool in a desired position; and
(g) utilizing the secured tool to carry out treatment procedures.

17. The method of claim 16 further including the step of repeating the previous steps as needed and completing fixation using tools and implants from a minimally disruptive fixation system as needed.
